(19) United States
(12) Patent Application Publication
(10) Pub. No.: US 2025/0228505 A1
(43) Pub. Date: Jul. 17, 2025

(54) MEDICAL SYSTEM, PORTABLE DISPLAY, METHOD FOR OPERATING A MEDICAL SYSTEM AND/OR A PORTABLE DISPLAY AND METHOD

(71) Applicant: KARL STORZ SE & Co. KG, Tuttlingen (DE)

(72) Inventors: Clemens BÜHLER, Tuttlingen (DE); Irina GERLACH, Tuttlingen (DE)

(73) Assignee: KARL STORZ SE & Co. KG, Tuttlingen (DE)

(21) Appl. No.: 19/016,539

(22) Filed: Jan. 10, 2025

(30) Foreign Application Priority Data
Jan. 12, 2024 (DE) 10 2024 100 941.1

(51) Int. Cl. A61B 5/00; A61B 5/0205; A61B 90/50

(52) U.S. Cl. CPC A61B 5/7445; A61B 5/0205; A61B 5/7282; A61B 5/7405; A61B 5/7455; A61B 5/746; A61B 2090/502

(57) ABSTRACT

The present disclosure relates to a medical system comprising a patient monitoring unit which is designed to record a vital sign of a patient, a portable display which can be worn on the head of a user and which, when worn, provides a user with an at least substantially unobstructed view of an environment within a field of view of the user, wherein the portable display is adapted to display, in the worn state, a vital sign information overlay in the field of view of the user based on the one vital sign parameter, and a patient condition detection unit adapted to determining at least one patient condition parameter relating to a current vital condition of the patient, wherein the portable display is adapted to display or not display the vital information overlay depending on the patient condition parameter.

### CROSS-REFERENCE TO RELATED APPLICATIONS

This application claims priority of German Patent Application No. DE 10 2024 100 941.1 filed on Jan. 12, 2024, the contents of which are incorporated herein.

### TECHNICAL FIELD

The present application relates to a medical system, to portable displays, to a method for operating a medical system and/or a portable display, and to a method.

### BACKGROUND

Nowadays, medical professionals are supported by a variety of systems when carrying out diagnostic, therapeutic and/or surgical procedures. With the continuous advancement of technology and more cost-effective manufacturing methods, the number of available systems is constantly increasing. Numerous devices are used in operating theaters, for example, including surgical microscopes, anesthesia machines, ventilators, surgical instruments, mobile X-ray machines, ultrasound machines, patient monitoring units, navigation systems and many more. Many of these machines have their own display units and provide medical professionals with visual information about the patient. During an operation, for example, in time-critical situations a surgeon performing the operation must identify those display units from the plurality of display units that are currently relevant and, at the same time, filter the information currently required out from a large amount of information.

For example, during the procedure, the surgeon can turn their focus and gaze to an incision site of a patient to visually monitor the progress of the procedure and quickly identify potential complications. However, if the surgeon requires information about the patient's vital condition, they must look away from the incision site and focus on a display device that provides the relevant vital condition information. This places greater strain on the surgeon's concentration as they must constantly change their focus. This can have serious consequences, errors may occur and the safety of the procedure may be compromised, especially during complex procedures that may last several hours.

In addition, the patient's condition may suddenly deteriorate during the procedure. The inventors have recognized that it is precisely in these time-critical situations that the surgeon should devote a considerable amount of their attention to the procedure itself. However, according to the prior art, the surgeon performing the operation must filter out and process important information about the patient's condition from the plurality of display devices within a short period of time.

As another example, complications may arise during intubation for establishing and maintaining the airways. During intubation, for example after an accident, blood and swelling can make intubation considerably more difficult. In addition, the patient may be in a critical condition. Information about the patient's condition can be crucial to the outcome of the procedure. For example, the surgeon may need information about the patient's blood oxygen saturation or pulse. However, if the surgeon has to obtain this information from a plurality of display units in a hectic situation, his focus may be diverted from actually performing the intubation and the quality with which the procedure is carried out may decrease.

The inventors have recognized that a user should be supported when obtaining and processing information about a patient. The information should be provided in a more targeted manner appropriate to the situation. In general, strain on the user should be relieved so that they can better concentrate on caring for the patient.

### SUMMARY

Proceeding from the prior art, the object addressed by the disclosure is that of efficiently providing information about a patient.

The object is achieved according to the disclosure by a medical system, by portable displays, by a method for operating a medical system and/or a portable display and by a method as described herein and defined in the claims. In addition, further aspects of the disclosure which explain the disclosure are described.

In one aspect, the present disclosure may provide the provision of a medical system. The medical system comprises a patient monitoring unit configured to record a vital sign of a patient, a portable display that can be worn on a user's head and that, when worn, provides a user with an at least substantially unobstructed view of an environment within a field of view of the user, wherein the portable display is configured to display vital information, which relates to the one vital sign, in the user's field of view when worn and a patient condition detection unit configured to determine at least one patient condition parameter that relates to a current vital condition of the user. According to this aspect, the portable display is configured to either display or not display the vital information on the basis of the patient condition parameter.

According to a further aspect, the present disclosure provides providing a portable display for a medical system according to the previous aspect.

According to a further aspect, the present disclosure provides providing a portable display, in particular for a medical system according to any one of the previous aspects. The portable display is wearable on a user's head and, when worn, provides a user with an at least substantially unobstructed view of an environment within a field of view of the user. The portable display is configured, when worn, to display vital information, which relates to the one vital sign, in the user's field of view. In addition, the portable display comprises a patient condition detection unit configured to determine at least one patient condition parameter relating to a current vital condition of the patient. According to this aspect, the portable display is configured to either display or not display the vital information on the basis of the patient condition parameter.

According to a further aspect, the present disclosure provides providing a method for operating a medical system according to any one of the preceding aspects and/or a portable display according to any one of the preceding aspects.

According to a further aspect, the present disclosure provides providing a method, in particular carried out by means of a medical system according to any one of the previous aspects and/or a portable display according to any one of the previous aspects. The method comprises the steps of recording a vital sign of a patient, providing a portable display for a user which can be worn on a user's head and which, when worn, provides the user with an at least substantially unobstructed view of an environment within a field of view of the user, displaying vital information, which relates to the vital sign, in the user's field of view by means of the portable display when the display is worn, determining a patient condition parameter that relates to a current vital condition of the patient, and displaying or not displaying the vital information on the basis of the patient condition parameter.

These features allow information about a patient to be provided efficiently. The information is provided in a more targeted, needs-based and situation-specific manner. Vital information is made available to the user, for example if it is worthy of note or should be taken into account in connection with the vital condition. Overall, less information may be provided to the user, but the information provided at a given time may be of primary importance. The vital information provided can change dynamically depending on the patient's condition. The user can be notified of this change, especially in a situation-specific manner, by displaying the relevant vital information. The user does not have to stop focusing on a procedure that is currently being performed, but instead is shown the vital information in their field of vision by means of the vital information displayed. As a result, less information is therefore provided for processing by the user, the user does not have to carefully monitor changes in the patient's condition on various display devices, and can concentrate better on a procedure that is to be performed at present. This puts less strain and demand on the user's concentration and focus. This allows higher quality to be achieved when performing the procedure, such as an operation or intubation. Patient care can be improved and the risk of treatment errors reduced.

According to a further aspect, the present disclosure provides providing a medical system. The medical system comprises a patient monitoring unit configured to record a vital sign of a patient, a portable display that can be worn on a user's head and that, when worn, provides the user with an at least substantially unobstructed view of an environment within a field of view of the user, wherein the portable display is configured to display vital information in the user's field of view when worn, which relates to the vital sign, and a field of view detection unit configured to determine at least one field of view parameter that relates to the user's current field of view. The portable display is configured to either display or not display the vital information on the basis of the field of view parameter.

According to a further aspect, the present disclosure provides providing a portable display for a medical system according to the previous aspect.

According to a further aspect, the present disclosure provides providing a portable display, in particular for a medical system according to any one of the previous aspects. The portable display is wearable on a user's head and, when worn, provides the user with an at least substantially unobstructed view of an environment within a field of view of the user. The portable display is configured, when worn, to display vital information, which relates to a vital sign, in the user's field of view. In addition, the portable display comprises a field of view detection unit configured to determine at least one field of view parameter relating to the user's current field of view. Furthermore, the portable display is configured to display and/or not display the vital information on the basis of the field of view parameter.

According to a further aspect, the present disclosure provides providing a method for operating a medical system according to any one of the preceding aspects and/or a portable display according to any one of the preceding aspects.

According to a further aspect, the present disclosure provides providing a method, in particular carried out by means of a medical system according to any one of the previous aspects and/or a portable display according to any one of the previous aspects. The method comprises the steps of recording a vital sign of a patient, providing a portable display for a user which can be worn on a user's head and which, when worn, provides the user with an at least substantially unobstructed view of an environment within a field of view of the user, displaying vital information in the user's field of view by means of the portable display when the display is worn, which relates to the vital sign, determining a field of view parameter that relates to the user's current field of view, and displaying or not displaying the vital information on the basis of the field of view parameter.

These features allow information about a patient to be provided efficiently. The information is provided in a more targeted, needs-based and situation-specific manner. The user is provided with information about the patient on the basis of the current field of view. The field of view is detected and vital information is or is not displayed on the basis of the field of view. For example, superfluous and/or redundant information can be prevented from being provided. In situations where the current field of view would render it unnecessary or even detrimental to display vital information, it is possible for said information not to be displayed. Again, it can be displayed in situations where the display of vital information is helpful. Vital information can be dynamically shown or hidden depending on the current field of view. The user is thus supported by the display of vital information so that they can concentrate better on a procedure that is currently underway. The information is easier to record and process. In addition, the user does not necessarily have to look at the patient monitoring unit to obtain vital information about the patient. This is displayed in the their field of vision depending on the field of vision. Consequently, convenience for the user is increased and the attention and/or focus required to obtain the vital information is reduced. This allows higher quality to be achieved when performing the procedure, such as an operation or intubation. Patient care can be improved and the risk of treatment errors reduced.

According to a further aspect, the present disclosure may provide providing a medical system. The medical system comprises a patient monitoring unit configured to record a vital sign of a patient, a portable display that can be worn on a user's head and that, when worn, provides a user with an at least substantially unobstructed view of an environment within a field of view of the user, wherein the portable display is configured to display vital information, which relates to the one vital sign, in the user's field of view when worn and a procedure step detection unit configured to determine at least one procedure step parameter that describes which step of a plurality of steps of a procedure to be performed by the user on the patient the user is currently performing. The portable display is configured to display and/or not display the vital information on the basis of the procedure step parameter.

According to a further aspect, the present disclosure provides providing a portable display for a medical system according to the previous aspect.

According to a further aspect, the present disclosure provides providing a portable display, in particular for a medical system according to any one of the previous aspects. The portable display is wearable on a user's head and, when worn, provides a user with an at least substantially unobstructed view of an environment within a field of view of the user. The portable display is configured to display vital information, which relates to the one vital sign, in the user's field of vision when worn and comprises a procedure step detection unit configured to determine at least one procedure step parameter that describes which step of a plurality of steps of a procedure to be performed by the user on the patient the user is currently performing. The portable display is configured to display and/or not display the vital information on the basis of the procedure step parameter.

According to a further aspect, the present disclosure provides providing a method for operating a medical system according to any one of the preceding aspects and/or a portable display according to any one of the preceding aspects.

According to a further aspect, the present disclosure provides providing a method, in particular carried out by means of a medical system according to any one of the previous aspects and/or a portable display according to any one of the previous aspects. The method comprises the steps of recording a vital sign of a patient, providing a portable display for a user that can be worn on a user's head and that, when worn, provides the user with an at least substantially unobstructed view of an environment within a field of view of the user, displaying vital information, which relates to the vital sign, in the user's field of view by means of the portable display when the display is worn, determining a procedure step parameter that describes which step of a plurality of steps of a procedure to be performed by the user on the patient the user is currently performing, and displaying or not displaying the vital information on the basis of the procedure step parameter.

These features allow information about a patient to be provided efficiently. The information is provided in a more targeted, needs-based and situation-specific manner. The user is purposefully provided with the vital information needed in connection with a specific procedural step. The total information provided at a given time can be reduced and only the most important information can be provided. The user themselves has to concentrate less on isolating and/or processing the information that is important at that time from a large amount of information. This reduces the strain on the user and allows them to focus better on the procedure to be performed. For example, according to the prior art, the user would have to concentrate on a new display device on which vital information can be provided for each new procedural step and select it from a display devices arranged in the region around them. However, the inventors have recognized that the strain on the user can be reduced if the information necessary for the corresponding step is recognized, preselected and displayed in the user's field of vision. Information of lesser importance can be hidden to reduce strain on the user. This allows higher quality to be achieved when performing the procedure, such as an operation or intubation. Patient care can be improved and the risk of treatment errors reduced.

The various aspects mentioned above allow for improvements in the provision of a patient's vital information.

When a system, device, unit and/or component is described in connection with different aspects of the present disclosure, it may in principle be the same system, device, unit and/or component. This may mean, for example, that a medical system may have features of different aspects described herein. Likewise, a portable display may have features of different aspects described herein. In other words, a medical system may combine the functions and associated benefits of some and/or all of the medical systems according to the aspects described herein.

The medical system may comprise a plurality of devices, units and/or components. The medical system can be intended for mobile and/or stationary use. For example, the medical system can be arranged and/or mounted in an ambulance, portable such that an emergency medical technician can carry it to a patient in an emergency kit or the like and made available to the patient on site, and/or may be provided in an operating theater or a treatment room. Furthermore, the medical system can be expandable, for example by adding additional patient monitoring units and/or expanding an existing patient monitoring unit. Furthermore, the medical system can be at least partially integrated into and/or at least partially formed by existing equipment of ambulances, emergency kits, operating rooms and/or. In this sense, the medical system can extend the functions of existing systems and/or provide additional functionality in addition to these systems.

Vital parameters can include measurable physiological condition variables that provide information about the patient's condition and/or functions. Vital parameters can be crucial for monitoring vital functions and changes therein. In principle, vital signs allow for an assessment of a patient's health and/or vital condition. Especially within the context of a procedure, monitoring vital signs can be crucial, as a change in these can indicate a critical situation. Furthermore, a change in a vital sign can provide information about a patient's reaction to a procedure. For example, if the patient is intubated, the vital sign ‘oxygen saturation’ can be used, for example, to check if the intubation procedure has been successful and/or to assess whether a critical situation could arise during the procedure. It is therefore possible to assess whether the tube has been inserted correctly, for example. Examples of vital signs include heart rate, respiratory rate, blood pressure, body temperature, oxygen saturation and/or carbon dioxide saturation in the blood, carbon dioxide concentration in respiratory air, electrical brain activity, intracranial pressure and many more. In particular, a plurality of procedure-specific vital signs may exist, such as those that are relevant in connection with a procedure performed on the liver and/or the brain, for example.

A procedure may, for example, refer to a therapeutic, diagnostic and/or surgical measure. In other words, the procedure may include a surgical procedure, an endoscopic examination, life-saving measures in emergency medicine and/or the like, for example.

The patient monitoring unit may comprise one or more, in particular medical, appliances and/or systems, each of which is configured to record vital signs and other important physiological information of a patient. In particular, the patient monitoring unit can be used to record at least one vital sign during the procedure. This means that the patient's condition can be monitored by means of a patient monitoring unit, at least substantially in real time and/or with a short time delay. A patient monitoring unit can be designed as a mobile, portable medical appliance and/or system. This may mean that a user can carry the patient monitoring unit to the patient where they use it to record vital signs. Furthermore, the patient monitoring unit can also be permanently mounted in an operating room, for example. Alternatively or additionally, the patient monitoring unit may comprise a set of appliances and/or systems designed to record vital signs. A patient monitoring unit often comprises a plurality of modular medical appliances that are installed, for example, on an equipment tower and can be moved around the operating room by this equipment tower. Additional modules can be added to the patient monitoring unit as required. Examples of such corresponding modules, appliances and/or systems may be an ECG appliance, a pulse oximeter, a blood pressure monitor, a respiratory rate monitor, a thermometer, an intracranial pressure monitor and/or an EEG appliance.

The patient monitoring unit may comprise a plurality of display devices, each of which displays only some of the vital signs or on which an image relating to a vital sign can be displayed. For example, each module, appliance and/or system of the patient monitoring unit may comprise its own display device on which only one vital sign recorded by the corresponding module, appliance and/or system can be displayed. Therefore, in order to obtain information about a particular vital sign, the user must select the correct display device from the plurality of display devices. This can lead to a critical loss of time and/or increased strain on the user, especially in hectic situations, emergencies and/or the like.

Furthermore, the patient monitoring unit can comprise a central display device on which a plurality of, and/or at least substantially all, the vital signs recorded can be displayed. In other words, information recorded by the individual modules, appliances and/or systems can be collectively made available on the central display device.

As already described, the medical system can be used, for example, in a clinical environment in an operating room. However, the medical system can also be usable in emergency situations in intensive care, for example in an ambulance or at the scene of an accident for providing first aid, for example. The user in this context may include medical professionals, in particular surgeons, anesthesiologists, emergency medical technicians and/or the like.

In general, the portable display can make information visually available to the user. This can be achieved, for example, by projecting an image, in particular directly, onto the retina. Furthermore, an image can also be displayable on a display. In particular, the display can be transparent. This may mean that the user can see through the display. The display can be formed integrally with a spectacle lens and/or arranged in the manner of a spectacle lens in the standing line of the user, for example. The portable display may include virtual reality (VR) glasses, augmented reality glasses, a retina projector, a head-mounted display, video glasses, a helmet-mounted display, and/or the like. In principle, common methods and/or appliances that are suitable for virtually extending what a user can see can be usable as portable displays. By means of the portable display, a real environment seen by the user can therefore be virtually extendable.

“Portable” can, in principle, mean that the display can be held and/or moved by the user's physical strength alone and is attached directly to the user. The display can therefore be used as a mobile display and can be easily movable by the user when worn. This may mean that the portable display is light enough for the user to wear comfortably for extended periods of time and, in particular, that wearing it does not unnecessarily restrict the user. The user can wear the portable display especially while performing a procedure. The portable display primarily has a supporting effect and does not restrict the user, particularly through its weight or spatial extent. In this context, “when worn” may refer to a state in which the portable display is attached to the user's head and arranged such that information can be made visually available.

The field of view can refer to a region that the user can simultaneously see with one and/or both eyes without moving their head and/or eyes. This may mean that the field of view includes what the user is currently seeing. In particular, the field of view can refer to a region that the user can see clearly and/or sees in focus. A periphery can be distinguished from this region in that the user only sees out of focus and/or can see at least substantially no details.

A “substantially unobstructed view of an environment within a field of view of the user” can be understood to mean that the user can view the environment without significant disturbances or limitations. In particular, this may mean that the environment can be viewed without disturbing elements and/or distractions, allowing the user to apply natural viewing behavior. The portable display can therefore be usable intuitively and does not restrict the user's field of vision. Rather, the portable display is designed to add additional information to the field of view. The user is not restricted in terms of their natural view of the real world.

By displaying the vital information, the vital information can be made visible to the user. The vital information displayed can virtually expand what the user can see. “Making it visually available” can be understood to mean that the user can read a quantitative value of the vital sign from the vital information displayed. The vital information displayed can comprise an image. For example, the vital information displayed may comprise an image of a number that corresponds to a quantitative value of a vital sign. Furthermore, displaying the vital information can also include an image of a pseudo-quantitative profile of a vital sign. “Relating to the vital sign” can also be understood as an image of information abstracted from one or more vital signs, a relationship between different vital signs and/or the like. For example, an image of a symbol that changes size depending on a quantitative value of a vital sign may include vital information being displayed that relates to the vital sign.

The vital information displayed can relate to a single vital sign and/or a plurality of different vital signs. According to some embodiments, a plurality of pieces of vital information may be displayed, each piece of vital information displayed at least partially relating to at least one different vital sign. The pieces of vital information can be displayable at the same location and/or at different locations in the user's field of vision.

In particular, the vital information can be displayable in the user's field of vision in such a way that it is substantially seamlessly integrated into the real environment in order to enable an unobstructed view of the environment within the user's field of vision. The user can perceive the displayed vital information conveniently and/or at least substantially without effort. In particular, this may mean that the vital information can be displayed in such a way that it is clearly visible to the user in a sharp field of vision of the user. An automatic adjustment of a virtual image plane of the vital information displayed may be made in order to ensure that the vital information displayed is clearly focused for the user. This can avoid and/or reduce visual fatigue and/or discomfort. The user can use the portable display intuitively and perceive the vital information displayed in a kind of casual manner. The vital information can be displayable in such a way that the user predominantly and/or primarily perceives the environment in their field of vision. However, the vital information can be displayable in the user's sharp field of vision, or in a region of the field of vision in which the user can clearly see the environment. For example, the vital information can be displayable in an edge region, or at the sides, of the field of view, in particular the sharp field of view. The user can then primarily direct their gaze to a patient, for example to monitor a procedure they are performing themselves, and to record the vital information using the vital information displayed in the edge region of the field of view without having to look away from the patient. This may mean that the user can clearly see the vital information displayed without accommodating their eyes to the vital information displayed.

Furthermore, the vital information displayed also does not obscure any important details of the environment that could be important for performing the procedure. The vital information displayed can be adaptable to the user's field of vision in such a way that it fits into the field of vision and is perceptible, in particular, without separately focusing on the vital information displayed. The vital information displayed can be adapted to different lighting conditions. This makes it possible to implement an application with an at least substantially consistent level of quality in different environments. User-friendliness can be ensured by ensuring that the vital information displayed does not interfere with the user's natural view of the real environment. In general, an immersive user experience can be achieved by the way in which vital information is displayed.

The field of view detection unit can detect the user's field of view and/or determine which part of the environment the user sees. In particular, this may include determining the viewing direction of the user and/or recording the user's surroundings, in particular the surroundings in the viewing direction. It is possible to record where the user is looking and/or, at least substantially, what they can see.

A field of view parameter can mean a quantitative value that is suitable for describing the detected field of view. This means that a field of view can be describable, for example, in a coordinate system and/or the field of view can be comparable with other parameters. Furthermore, the field of view parameter can be processable, for example by means of an electronic data processing means. It can therefore carry information used to describe the field of view, in particular mathematically and/or geometrically. For example, the field of view detection unit can be used to measure/determine an eye movement of the user and/or to infer the field of view using the measured eye movement and/or, in particular, to determine a relative change in the field of view. Furthermore, the field of view parameter can be used to describe the contents of the field of view.

The field of view detection unit may be at least partially based on an eye tracking method and/or implement an eye tracking method. The field of view detection unit may, for example, comprise an infrared light source configured to shine infrared light onto the user's eyes. Furthermore, the field of view detection unit can comprise cameras, in particular infrared cameras, and/or infrared sensors, which are designed to spatially capture light reflected from the eyes, in particular infrared light. Using image processing algorithms, the user's viewing direction can be tracked and/or determined using the captured light, particularly infrared light. Furthermore, the field of view detection unit can comprise a camera which is configured to image the user's surroundings, in particular at least substantially in the user's viewing direction, and to generate image data. This image data can carry information about the contents of the user's field of view. Other procedures and/or units may also be meant by the field of view detection unit. The procedure described above is to be understood as an example only.

A field of view parameter that is determined may include a viewing angle and/or a viewing cone. This can be resolved spatially, i.e. in three spatial directions. The field of vision can be describable by means of the viewing angle and/or the viewing cone. Furthermore, the field of view parameter may include a position of at least one eye of the user. In general, a field of view parameter can include a vector description of the field of view and/or the viewing direction in a reference coordinate system. The reference coordinate system can be used to describe the user's position in space. The reference coordinate system may provide a reference for defining the spatial location and/or position of the user and/or the environment.

Furthermore, the field of view parameter can contain information about which part of the environment the user can see clearly. In this context, the field of view parameter can be used to describe an extent and/or range of the user's field of view, or the field of view parameter can include a quantitative value in this regard.

In addition, the field of view parameter may comprise information about the user's environment. For example, the field of view parameter may comprise image data based on an image of the environment. For example, image detection algorithms can be applied to determine information about details that are clearly seen by the user on the basis of the field of view parameter. Results of an image recognition algorithm can also be transmittable by the field of view parameter. In principle, it is thus possible to at least substantially determine where the user looked and what they saw. In this context, “relating to the current field of view” may mean that information about what the user can see according to the field of view detection unit is collected by means of the field of view parameter. The field of view parameter may comprise information about details that are arranged in the user's field of view but are not perceptible to and/or consciously perceived by the user. For example, the field of view parameter may comprise information about the contents of an image in the user's field of view, wherein the user does not primarily direct their gaze to this image.

“To either display or not display on the basis of a parameter” can mean that an action and/or decision can be based on the parameter, in particular a value and/or information content of the parameter. The parameter can at least partly determine whether vital information is displayed or not. In some embodiments, the mere presence of a parameter may cause the vital information to be or not to be displayed. In other embodiments, a value of the parameter may cause the vital information to be or not to be displayed. A decision whether or not to display the vital information may be based on a mathematical calculation rule and/or an algorithm, for example using artificial intelligence, machine learning, deep learning and/or a neural network, in particular an open, closed, single-layer and/or feedback neural network and/or a combination thereof.

In this context, “on the basis of the field of view parameter” may mean, for example, that the vital information is or is not displayed on the basis of the field of view and/or the contents of the user's field of view. This may mean, for example, that when the user tilts their head, looks up/down, looks to the side and/or the like, the information is either displayed or is not displayed. It is conceivable that the vital information is displayed in a zero position in which the user is looking at the patient. However, in a position deviating from the zero position, the vital information is not displayed. Furthermore, it is, for example, possible to detect whether the user is not currently performing a procedure, for example. In this case, it is possible for the vital information not to be displayed.

Detection units described herein may also be based on a mathematical calculation rule and/or an algorithm, for example using artificial intelligence, machine learning, deep learning and/or a neural network, in particular an open, closed, single-layer and/or feedback neural network and/or a combination thereof.

In general, the patient monitoring unit can be designed to record different vital signs. The portable display can then be configured to display a plurality of different pieces of vital information in the user's field of vision when worn and/or to use the different vital signs as the basis for the vital information displayed.

This can be done in particular on the basis of the field of view parameter. This may mean that one of the pieces of vital information displayed is made and another piece of the vital information is or is not displayed on the basis of a parameter, in particular the field of view parameter. Alternatively or additionally, information about another vital sign can be visually displayed in the same vital information on the basis of a parameter, in particular the field of view parameter.

The patient condition detection unit can in particular be configured to determine the condition of the patient using at least one vital sign. The condition can mean in particular the vital condition and/or the general current condition of the patient. This can be defined using the vital signs. For example, the patient may have a heart rate of 80 beats per minute in a first vital condition and 160 beats per minute in a second vital condition. The patient condition detection unit is designed to detect changes in this vital condition. In some embodiments, the patient condition detection unit is configured to track a profile of at least one vital sign and to determine the vital condition on the basis of the profile. Furthermore, the patient condition detection unit can be configured to compare the at least one vital sign with limit values and to determine the vital condition using a result of the comparison. Alternatively or additionally, the patient status detection unit can be configured to determine the vital condition from a plurality of vital signs. For example, the vital signs can be compared and/or placed in a relationship. Quotients can be formed and/or comparisons with reference values can be made. In particular, the patient condition detection unit is designed to detect abnormalities in the vital condition. In general, the patient condition detection unit can be used to detect whether vital information, in particular relating to a specific vital sign, needs to be displayed, helpful and/or important, or whether the display of vital information is more likely to have a disruptive effect on the user.

The vital condition can in principle refer to a patient's current physiological condition. The vital condition can refer to a set of physiological parameters and/or vital signs. In some embodiments, the vital condition may refer to the vital signs that are most important for the patient's survival. These may include heart rate, blood pressure, respiratory rate and body temperature, for example.

By means of the patient condition parameter, which refers to the patient's current vital condition, the vital condition can be quantified and/or made comparable. In some embodiments, the patient condition parameter may comprise and/or relate to a vital sign. The patient condition parameter may comprise a quantitative value by which the vital condition can be assessed. In some embodiments, the patient condition parameter may be used to infer a vital sign that is of primary importance to the user. The vital information displayed can relate to this vital sign.

By displaying vital information or not on the basis of the patient condition parameter, a user can be made aware of the patient's vital condition. For example, the vital information can be displayed if an anomaly in the vital condition has been detected using the patient condition parameter and/or by means of the patient condition detection unit. Furthermore, vital information can be displayed when a change in the vital condition is detected, for example using the patient condition parameter.

As already described, a procedure can, for example, refer to a therapeutic, diagnostic and/or surgical measure. In other words, the procedure may include a surgical procedure, an endoscopic examination, life-saving measures in emergency medicine and/or the like, for example. A procedure can comprise a complex sequence of actions that can be divided into a plurality of different steps to be performed by the user. By means of the procedure step parameter, the procedure step detection unit can provide a quantitative value that can be used to determine which step of the plurality of steps of the procedure to be performed by the user on the patient the user is currently performing. A comparative value can therefore be provided, on the basis of which the progress of the procedure and/or sequence of actions can be assessed and/or determined. If the procedure involves something like microinvasive laparoscopy, a first step may involve making an incision, a second step may involve preparing an endoscope, a third step may involve inserting the endoscope into the abdomen, and a fourth step may involve performing an imaging examination of the abdomen. The procedure step detection unit can be configured to detect which of these steps the user is currently at. In order to provide a comparative value by means of a computing unit, by means of which value the current step can be identified, the procedure step detection unit can be configured to provide and/or determine the procedure step parameter.

One of the steps may require vital information relating to a vital sign to be displayed more so than another of the steps. In other words, a certain piece of vital information may be more important to the user in one of the steps than a different piece of vital information. The portable display can take this into account when displaying the vital information or not. For example, when the incision is performed, it may be important to display information about blood pressure. For example, oxygen saturation may be of secondary importance. However, if the imaging examination is performed, oxygen saturation may be of primary importance. The vital information displayed can then relate to oxygen saturation and information about blood pressure may not be displayed.

Additionally, the field of view parameter may include information regarding the contents of the user's field of view. Advantageously, vital information may or may not be displayed on the basis of the contents of the field of view. For example, information regarding the contents of the field of view may include people, objects and/or the like. In general, the information regarding the contents can include everything that the user can see.

The medical system may further comprise a display device configured to display a vital information image that relates to the vital sign, wherein the portable display is configured not to display the vital information if the field of view parameter indicates that the display device is in the user's field of view. Advantageously, this can prevent the vital information displayed from providing disadvantageous information in certain situations. It can be ensured that the user is not distracted by the display of the vital information. Furthermore, it can be ensured that information is not provided multiple times. For example, it can be detected that the user is currently looking at a heart rate monitor that displays the pulse. It is then possible for vital information relating to the pulse not to be displayed.

Furthermore, the medical system may comprise a medical imaging instrument configured to generate image data and transmit the image data. In addition, the medical system may comprise a display device configured to receive the image data and to generate an image for a user. The portable display may be configured not to display the vital information if the field of view parameter indicates that the display device is in the user's field of view. Advantageously, the user can be prevented from being distracted by the vital information being displayed when looking at the display device. For example, the medical imaging instrument may comprise an endoscope and the user may perform an endoscopic examination on the patient. The user views the image relating to the endoscopic image data on the display device. Displaying vital information could be disadvantageous since the user should primarily focus on the endoscopic examination.

In addition, the portable display may be configured to display the vital information when the field of view parameter indicates that the patient is in the user's field of view. Advantageously, the user has relevant vital information in their field of vision when looking at the patient in order to be able to make a quick, efficient and easy assessment of the patient's vital condition. For example, the field of view parameter may comprise information relating to the contents of the field of view, according to which the user is currently looking at the patient. In such a case, vital information can be displayed. However, if it is detected that the user is not looking at the patient and the field of view parameter accordingly comprises information that the patient is not in the user's field of view, the vital information may not be displayed. It is conceivable, for example, to use the portable display and/or the medical system during a doctor's visit in a hospital. The doctor can go from one patient to the next and is shown the relevant vital signs when looking at the new patient. This can save a lot of time.

A particularly mobile and user-friendly medical system can be provided if the field of view detection unit is arranged on the portable display. For example, the portable display may be configured to wirelessly receive information regarding the vital sign and/or the vital information displayed. A compact system can be provided.

Alternatively or additionally, the field of view detection unit may be arranged separately from the portable display. This may provide a particularly lightweight portable display device and/or allow the field of view detection unit to determine more precise and/or accurate field of view parameters. For example, the field of view detection unit may comprise a camera system arranged in an operating room. Furthermore, the field of view detection unit can be arranged on the patient monitoring unit. This makes it quick and easy to determine whether the user is currently looking at the patient monitoring unit.

According to some embodiments, the field of view detection unit is configured to determine the viewing direction of the user. The field of view parameter can relate to the viewing direction that is determined. This means that the decision as to whether or not to display vital information can relate to the user's viewing direction. In particular, a change in the viewing direction can be identified and the vital information displayed can relate to the change. If the user looks to one side, for example. the vital information may or may not be displayed.

In principle, the portable display can be integrable into optical corrective glasses. This allows users who are visually impaired to use the portable display. The portable display can comprise an attachment, for example, in particular by means of a projector and/or retina projector, for the corrective glasses. Furthermore, an integral design of a corrective lens having a display is also conceivable.

In addition, the portable display may include an alarm device configured to alert the user when at least one of the vital signs is in a critical range. In critical situations, attention can be increased quickly and efficiently and the user can be made aware of the critical situation. In particular, the patient condition detection unit can be used to determine whether one of the vital signs is in a critical range.

In addition, the portable display may be configured to display the vital information independently of the field of view parameter if at least one of the vital signs is in a critical range. An override function can be provided which, in critical situations, at least substantially inevitably provides the provision of information relating to critical vital signs. In emergency situations, relevant information is quickly and easily available to the user. In particular, the patient condition detection unit can be used to determine whether one of the vital signs is in a critical range.

Furthermore, the portable display can be configured to display the vital information if the at least one patient condition parameter is in a critical range. The portable display may be configured not to display the vital information if the at least one patient condition parameter is not in the critical range. The vital information can be displayed in a manner tailored to the specific situation and in particular in emergency situations. The user is not overloaded with information, but is provided in particular with information that is of primary importance. The critical range may comprise, for example, a range of values for a vital sign and/or a quantitative variable relating to at least one vital sign, above and/or below a limit value. For example, vital information relating to the heart rate can be displayed, if this is above 120 beats per minute. The critical range can also be adapted to suit the specific situation. For some procedures, for example, a heart rate of 120 beats per minute is not critical, but for other procedures it is. With regard to the patient condition parameter, a critical range may, for example, take into consideration a plurality of vital signs. This may mean that not only the heart rate is compared with a limit value. Instead, heart rate, oxygen saturation and respiratory rate can be collectively taken into account to establish whether the patient condition parameter is in a critical range.

In addition, the portable display may comprise an alarm device configured to alert the user when the at least one patient condition parameter is in a critical range. In critical situations, attention can be increased quickly and efficiently and the user can be made aware of the critical situation.

The user can be alerted to a critical situation, a critical vital sign and/or a critical patient condition parameter particularly efficiently and effectively if the portable display is configured to alert the user by means of a vibration alarm. For example, the portable display may comprise glasses, in particular AR glasses and/or VR glasses, having temples. A device for generating the vibration alarm can be arranged in the temples. In principle, it can be advantageous if a vibration device designed to provide the vibration alarm is integrated into a fastening of the portable display, wherein the fastening is designed to fasten the portable display to the user's head.

Alternatively or additionally, the portable display may be configured to alert the user by means of an acoustic alarm signal. An acoustic warning signal is easy to provide. In some embodiments, the portable display is configured to alert the user by means of a plurality of different alarm signals. For example, an acoustic alarm signal and a vibration alarm can be provided. At least one alarm signal can be providable in a way that corresponds to the urgency of the critical situation. If the patient's life is in danger, for example, the acoustic alarm signal can be louder.

In addition, the portable display can be configured to visually enhance the display of vital information on the basis of the patient condition parameter. This makes it particularly efficient for the user to be made aware of any changes in the patient's vital condition, for example. Such a method of gaining the user's attention is particularly reliable. The user immediately sees a change in the patient's vital condition during the procedure. In particular, this allows the user to quickly and easily establish a patient's reaction to a procedure. “Visual enhancement” may mean that the color and brightness of the vital information displayed is changed, the transparency is reduced, the contrast is increased, a background is changed, and/or the like.

Alternatively or additionally, the portable display may be configured to enlarge the vital information displayed on the basis of the patient condition parameter. Depending on the situation, vital information relating to a vital sign that is more important at that moment can be displayed larger, for example, thereby making the user notice it more.

In addition, the portable display can be configured to display the vital information in a manner in which it flashes, at least in part, on the basis of the patient condition parameter. A flashing image is perceived particularly reliably by the user. It can ensure that the user perceives vital signs that are important at that moment.

In addition, the portable display can be configured to display the vital information regardless of the user's viewing direction. This can ensure that vital information important at that time is displayed in at least substantially every case, or regardless of the viewing direction.

According to some embodiments, the patient monitoring unit may be configured to record different vital signs, wherein the portable display is configured to display, when worn, a plurality of different pieces of vital information in the user's field of view that relate to different vital signs, and wherein the portable display is configured to display at least one of the plurality of different pieces of vital information on the basis of the corresponding step of the procedure to be performed, which step is detected by the procedure step detection unit. Advantageously, information is provided about the vital sign that is of primary importance for performing the current step. The user is purposefully provided with information, thereby efficiently supporting them in carrying out the procedure. In this context, displaying vital information can be understood as displaying each vital sign. For example, vital information may be displayed for blood pressure and vital information may be displayed for pulse.

Furthermore, the portable display can be configured to visually enhance the vital information displayed on the basis of the procedure step parameter. Such a method of gaining the user's attention is particularly reliable. In a first step, it may be more important to display vital information than in a different step. In the first step, the vital information can therefore be displayed visually enhanced. The user immediately sees a change in the patient's vital condition during the procedure. In particular, this allows the user to quickly and easily establish a patient's reaction to a procedure. “Visual enhancement” may mean that the color and brightness of the vital information displayed is changed, the transparency is reduced, and/or the like.

In addition, the portable display may be configured to enlarge the vital information displayed on the basis of the procedure step parameter. For example, a piece of vital information that relates to a vital sign that is more important at that moment can be displayed larger for a specific step such that the user notices it more.

In addition, the portable display can be configured to display the vital information in a manner in which it flashes, at least in part, on the basis of the patient condition parameter. A flashing image is perceived particularly reliably by the user. It can ensure that the user perceives vital signs that are important at that moment.

In addition, the portable display can be configured to display vital information regardless of the user's viewing direction. This can ensure that vital information important at that time is displayed in at least substantially every case, or regardless of the viewing direction.

In addition, the procedure step detection unit can comprise a camera configured to generate image data, and the procedure step detection unit can be configured to determine the at least one procedure step parameter using the image data. This ensures safe and efficient step detection. The procedure step detection unit can comprise an image detection unit. This can be based on machine learning and/or comprise a neural network.

In addition, the camera can be arranged on the portable display. This makes it possible to provide a compact and/or mobile medical system. The camera can be arranged in a similar way to a camera for field of view detection. A common camera can be provided for field of view detection and procedure step detection. In general, the portable display may comprise a camera configured to generate image data of the user's surroundings, in particular in the user's viewing direction. Furthermore, the procedure step detection unit can be at least partially arranged on the portable display.

According to some embodiments, the camera may be arranged separately from the portable display. This may make it possible to determine the procedure step parameter independently of the user's viewing direction and/or the user's presence, for example. The patient can be monitored particularly safely.

According to some embodiments, the procedure may be the intubation of the patient. Advantageously, the user can be informed at each step of the intubation procedure about vital information relevant to the corresponding step. This can be particularly relevant when assessing whether a tube that was intended to be fed into the trachea was actually fed therein. It may happen that the tube is mistakenly inserted into the esophagus. In such a case, for example, oxygen saturation would drop and abnormalities would arise during a respiratory gas analysis. For example, the user can be informed, within their field of vision, whether the tube has been inserted correctly while switching on the ventilator. The user does not have to look away from the patient, but can observe the patient while ventilation begins and at the same time receive information about the corresponding vital signs.

The medical system may further comprise a patient condition detection unit configured to determine at least one patient condition parameter relating to a current vital condition of the patient. Advantageously, several aspects described herein can be combined to provide a particularly high-performance medical system.

In this context, the portable display may be configured to display or not display the vital information on the basis of the procedure step parameter and the patient condition parameter. Patient safety can be improved by taking into account the current step and also checking whether the vital condition is deteriorating. The user can be informed and/or supported in a targeted and efficient manner by displaying appropriate vital information.

In addition, the portable display may be configured, when the patient condition parameter indicates a critical vital condition of the patient, to display or not display the vital information independently of the procedure step parameter. For example, in emergency situations, it can be ensured that the user receives information about all the necessary vital signs.

The present disclosure is described below by way of example with reference to the accompanying figures. The drawing, the description and the claims contain numerous features in combination. The person skilled in the art will also expediently consider the features individually and use them sensibly in combination within the scope of the claims.

If there is more than one example of a particular object, only one of these is marked with a reference symbol in the figures and in the description. The description of this specimen can be transferred to the other specimens of the object accordingly. If objects are named using numerical words in particular, such as first, second, third object, etc., these are used to name and/or assign objects. Accordingly, for example, a first object and a third object may be comprised, but not a second object. However, a number and/or a sequence of objects could also be derived from number words.

### BRIEF DESCRIPTION OF DRAWINGS

FIG. 1 shows a schematic view of a medical system;

FIG. 2 shows another schematic view of the medical system;

FIG. 3 shows a schematic view of a user's field of view;

FIG. 4 shows another schematic view of a user's field of view;

FIG. 5 shows another schematic view of a user's field of view;

FIG. 6 shows another schematic representation of the medical system;

FIG. 7 shows another schematic view of a user's field of view;

FIG. 8 shows another schematic view of a user's field of view;

FIG. 9 shows a schematic view of a portable display; and

FIG. 10 shows a schematic flow diagram of a method.

### DETAILED DESCRIPTION OF PREFERRED EMBODIMENTS

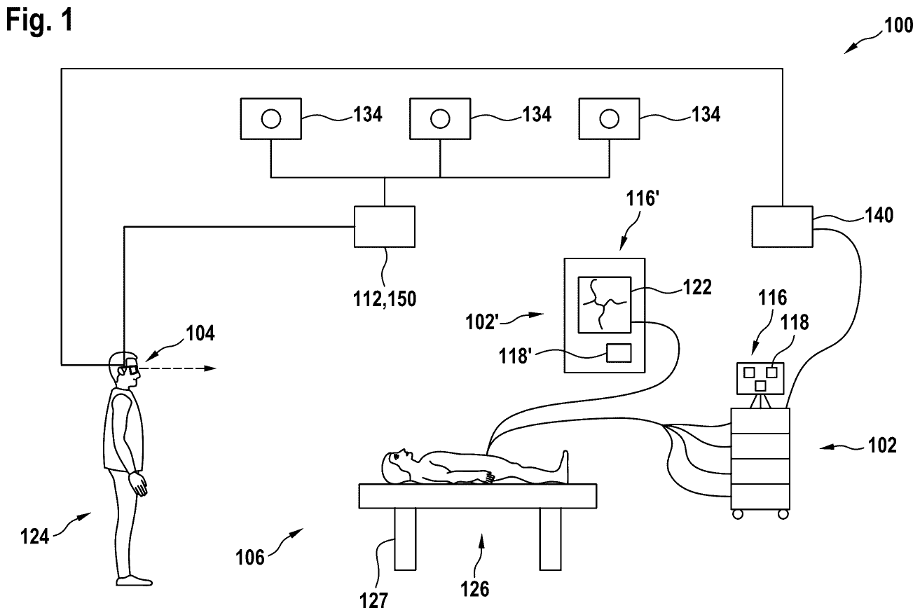

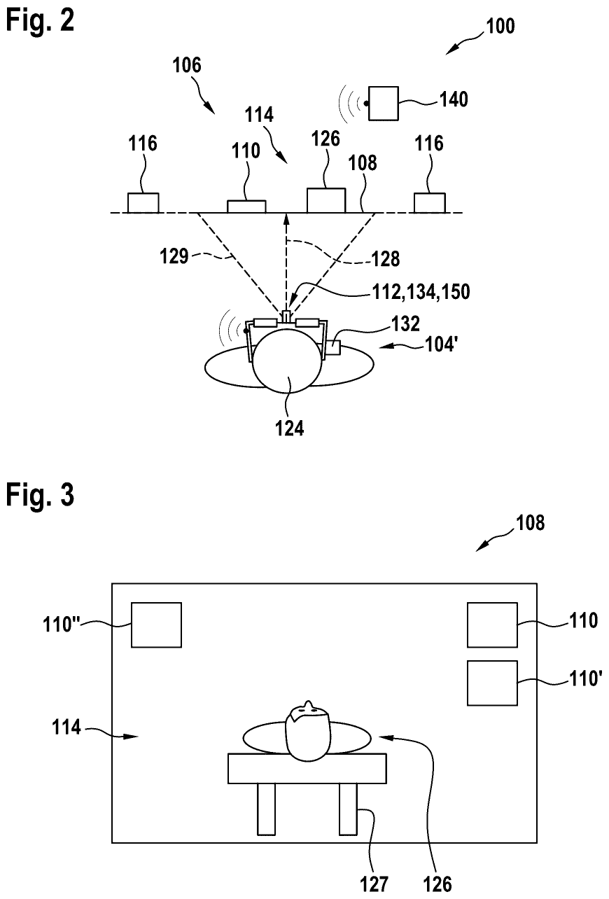

FIG. 1 is a schematic view of a medical system 100. A user 124 is shown wearing a portable display 104 on their head. According to FIG. 1, the portable display 104 comprises VR glasses and is configured to display vital information within a field of view 108 (see, for example, FIG. 2) of the user 124, thereby allowing an at least substantially unimpaired view of an environment 106 within the field of view 108 of the user 124. The vital information displayed can virtually extend the real environment 106 seen by the user 124 and in particular includes information about a vital condition of a patient 126. The portable display 104 is configured to display vital information 110, which relates to a vital sign, in the field of view 108 of the user 124 when being worn.

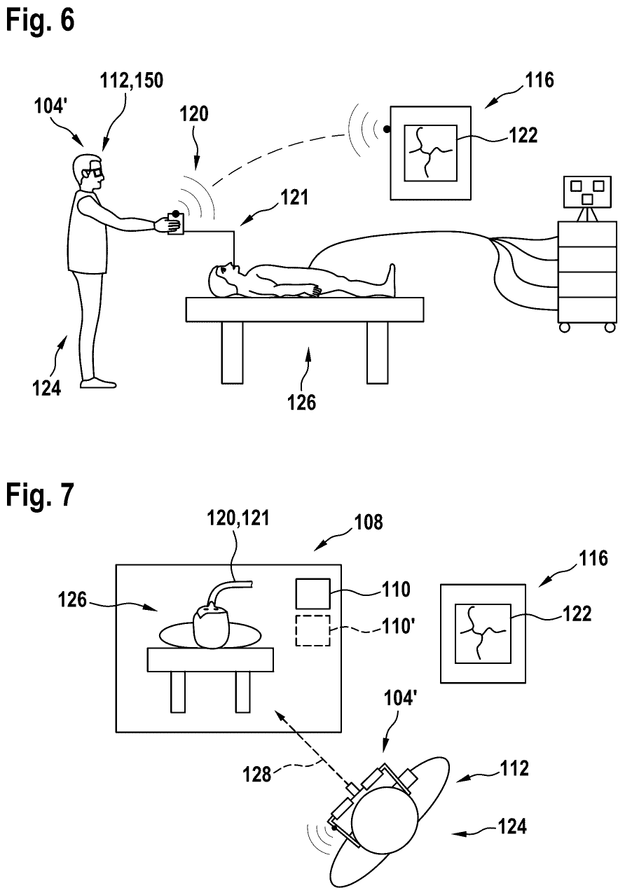

In FIG. 1, the medical system 100 is shown by way of example in a stationary application in an operating room. A mobile application in an ambulance or the like is also conceivable. The patient 126 lies on a treatment couch 127 and the user 124 performs a procedure on the patient 126. The procedure may include a surgical procedure, diagnostic activity and/or monitoring of the patient's vital condition. FIG. 6 shows a schematic view of the medical system 100, wherein the procedure is the intubation of the patient 126 by means of a video stylet.

The vital condition of the patient 126 is monitored by means of a patient monitoring unit 102. This is designed to record a plurality of different vital signs of the patient 126. The patient monitoring unit 102 comprises a plurality of modules for this purpose, each of which records at least one vital sign. For example, one module is a thermometer and records the patient's temperature. Another module is a pulse oximeter and records the oxygen saturation in the blood. Furthermore, the patient monitoring unit 102 comprises a display device 116 which is configured to display a vital information image 118 that relates to the vital sign. For example, a blood pressure profile, a patient's pulse, a respiratory rate and/or the like are displayed. The display device 116 is a central display device that is configured to display a plurality of vital information images 118 that relate to different vital signs.

In addition, at least one second patient monitoring unit 102′ is provided. To increase clarity, the reference signs for similar, related and/or recurring units are designated by apostrophes. The second patient monitoring unit 102′ also includes a display device 116′ on which a vital information image 118′ is displayed. In addition, an image 122 is generated on the display device 116′ for the user 124, which relates to image data from a medical imaging instrument 120 (see FIG. 6), in particular an endoscope. The display device 116′ may, for example, comprise a mobile monitor of an endoscope.

Information about the vital condition of the patient 126 and/or images relating to a vital sign can be displayed to the user within the field of view, as already described. The information may or may not be displayed as needed, depending on the situation and/or in a targeted manner, so that the user 124 is only provided with information about their field of vision 108 that is of primary importance. This will be explained in more detail below.

In order to determine which information is of primary importance to the user 124 at that moment, the medical system 100 comprises a field of view detection unit 112, a patient condition detection unit 140 and a procedure step detection unit 150. In other embodiments, only one or two of the units may also be provided. The units 112, 140, 150 may be intended to interact.

The field of view detection unit 112 is configured to determine at least one field of view parameter that relates to the current field of view 108 of the user 124. Using the field of view parameter, the portable display 104 can decide whether or not to display the vital information display 110 on the basis of the field of view parameter. The following drawings will be used to explain some examples of the basis on which the decision could be made and/or what information the field of view parameter could carry. In general, it is possible for the vital information not to be displayed if the user 124 is looking at one of the display devices 116, 116′ and an image 118 relating to the vital sign on which the vital information 110 displayed on the portable display 104 would be based is already displayed thereon. In other words, it is possible not to display a pulse of the patient 126 if an image relating to the pulse of the patient 126 is already in the field of view 108 of the user 124. This means that the same information is not provided multiple times. This means that the user is less distracted.

Conversely, it can also be seen that an image relating to the pulse is not provided within the field of view 108. Vital information can then correspondingly be displayed. This allows the user 124 to quickly and easily obtain information about the pulse without having to look at a display device showing a corresponding image. For example, this allows the user to concentrate better on an operation they are currently performing on the patient 126. In principle, it is conceivable that the vital information displayed is dependent on a viewing direction 128 of the user 124 and/or the contents 114 of the field of view 108. The contents 114 may include the patient 124 and/or one of the display devices 116, 116′, for example.

The field of view detection unit 112 may be arranged on the portable display 104 and/or separately from the portable display 104. In FIG. 1, three cameras 134 are shown which are configured to record image data for the environment 106, the user 124 and/or the patient 126. This allows spatial information relating to the user and/or their behavior o be obtained. The cameras 134 can be connected to the field of view detection unit 112 or be part thereof. The field of view detection unit 112 can use the spatial resolution to determine the viewing direction 128 of the user 124 and provide a field of view parameter relating to the viewing direction 128 determined. In general, the cameras 134 can be used to determine what the user 124 can see, and, on the basis thereof, the vital information can either be displayed or not.

The cameras 134 can also be connected to the procedure step detection unit 150 or be part thereof. The procedure step detection unit 150 and the field of view detection unit 112 can be collectively implemented on a computing unit. The procedure step detection unit 150 is configured to determine at least one procedure step parameter that describes which step of a plurality of steps of a procedure to be performed by the user 124 on the patient 126 the user 124 is currently performing. The portable display 104 is configured to display and/or not display the vital information 110 on the basis of the procedure step parameter.

As already described, the patient monitoring unit 102 is configured to record various vital signs. Based on the different vital signs, the portable display 104 may or may not display different vital information 110. Which vital information 110 is displayed is determined using the detected step of the procedure to be performed.

The procedure step detection unit 150 is configured to determine the at least one procedure step parameter using the image data generated by the cameras 134. The procedure is thus monitored and/or analyzed by means of the cameras 134. Based on the analysis result, the vital information 110 can be purposefully displayed or not.

A patient condition parameter can also be determined by the cameras 134. This is in principle determined by means of the patient condition detection unit 140, which can be configured to analyze image data from the cameras 134. The patient status parameter relates to a vital condition of the patient. It carries information about the physiological condition values of the patient 126. The portable display 104 is configured to display or not display the vital information 110 on the basis of the patient condition parameter. If the patient's vital condition and/or the patient condition parameter is within a critical range, for example because oxygen saturation has dropped significantly or blood pressure has increased significantly, the vital information can be displayed. However, if oxygen saturation or blood pressure moves within a normal physiological range, it is possible for the vital information not to be displayed. However, the patient condition detection unit 140 is primarily configured to analyze, compare and/or the like vital signs of the patient monitoring unit 102 in order to determine the patient condition parameter and in particular to establish whether the patient condition parameter is in a critical range or whether the patient's vital condition is in a critical range.

The decision whether to display the vital information or not can be made on the basis of all the parameters, i.e. the patient condition parameter, the field of view parameter and the procedure step parameter. For example, an algorithm can be used to decide whether or not to display the vital information. Furthermore, the medical system has an override function which causes vital information 110 to inevitably be displayed when a corresponding vital sign and/or other parameter is in a critical range. This can be done, for example, independently of the field of view parameter and/or the procedure step parameter. To alert the user, an alarm device 132 (see FIG. 2) is provided. The user can be alerted by means of a vibration alarm and/or an acoustic alarm signal.

As shown in more detail in FIGS. 3 to 5, the attention of the user 124 can be increasingly drawn to the vital information 110 displayed if it is flashing, visually highlighted, or enlarged. This can be done on the basis of at least one parameter described herein.

FIG. 2 is a further schematic view of the medical system 100. The head of the user 124 is shown schematically in a view from above. The user 124 is wearing a portable display 104′ that is very similar to the display 104 on their head. Instead of the display 104, the display 104′ and/or another portable display according to the disclosure can also be used. The portable display 104′ is provided as VR glasses.

The portable display 104′ comprises the field of view detection unit 112 and the procedure step detection unit 150. The field of view detection unit 112 is configured to determine a viewing direction 128 of the user 124. For this purpose, the field of view detection unit 112 comprises an infrared light source (not shown in detail) which is configured to illuminate the eyes of the user 124 with infrared light and at least one infrared sensor (not shown in detail) which is configured to spatially capture infrared light reflected by the eyes. By means of an image processing algorithm, the viewing direction 128 of the user 124 is tracked and/or determined using the captured infrared light. In addition, the field of view 108 of the user 124 can be determined and/or estimated by said algorithm and/or by other field of view detection methods. In particular, this can be done by determining a viewing cone 129.

Furthermore, the field of view detection unit 112 comprises a camera 134 which is configured to image the environment 106 of the user 124 and to generate image data. The camera 134 is aligned such that its optical axis is arranged at least substantially in parallel with the viewing direction 128 and/or extends at least substantially in the viewing direction 128. The camera 134, together with the viewing direction 128, can estimate the field of view 108 of the user 124. Furthermore, the field of view detection unit 112 is configured to determine what the user 124 is focusing their gaze on. This allows the contents of the user's field of view 108 to be determined. In addition, it is possible to determine what exactly the user 124 can currently see.

The image data generated by the camera 134 disposed on the portable display 104′ may thus carry information about the contents of the user's field of view. This image data can also be used by the procedure step detection unit 150 to detect the step of the procedure. The camera 134 can be considered to be part of the procedure step detection unit 150 and the field of view detection unit 112. The cameras 134 according to FIG. 1 can additionally be provided. However, the portable display 104′ is also particularly suitable for mobile applications.

Using the image data from the camera 134, the contents 114 in the field of view 108 of the user 124 can be determined. In this way, it is possible to establish that the patient 126 is located in the field of view 108. A corresponding field of view parameter is created and the portable display 104′ makes a decision on the basis of this field of view parameter as to whether to display the vital information 110 or not. According to FIG. 2, the vital information 110 is to be displayed when the patient 126 is in the field of view 108 of the user 124. Consequently, the vital information 110 is displayed. This is displayed in the field of view 108 of the user 124 in such a way that the user can clearly see the vital information 110 displayed when looking at the patient 126. FIG. 2 schematically illustrates this by the vital information 110 displayed and the patient 126 at least partially being in the same plane.

It can also be seen in FIG. 2 that display devices 116 are arranged in the environment 106 of the user 124. However, the field of view detection unit 112 detects that the user is not looking at the display devices 116 or that they are arranged outside the field of view 108. However, the camera 134 may be configured to at least partially image the environment 106 outside the field of view 108. It is therefore possible to detect at which angle relative to the viewing direction 128 a display device 116 is arranged. For example, the field of view parameter can then be determined on the basis of the viewing direction.

Furthermore, it can be seen in FIG. 2 that the portable display 104′ is wirelessly connected to the patient condition detection unit 140. Thus, the portable display 104′ can implement the above-described functions of the patient condition detection unit 140. In particular, the override function can be implemented, which causes certain vital information to be displayed in an emergency situation. To generate an alarm signal, the portable display 104′ comprises an alarm device 132. This comprises a microphone for generating an acoustic alarm signal and a vibration device for generating a vibration alarm. The vibration device is a arranged a bracket of the portable display 104′.

FIG. 3 is a schematic view of the field of view 108 of the user 124 looking at the patient 126 during a procedure. That which the user 124 can see is shown schematically. The patient 126 lies on the treatment couch 127, as also shown in FIG. 1. The field of view detection unit 112 detects the contents 114 of the field of view 108 and consequently detects that the patient 126 is within the field of view 108 of the user 124. In addition, it also detects that a display device is not in the field of view 116 on which vital information is displayed. Therefore, vital information 110, 110′, 110″ is displayed. These virtually extend the real environment of the user 124 and are seen by the user at the edge of the field of view 108. The vital information displayed 110, 110′, 110″ does not interfere with the user's view of the patient 126. For example, three items of displayed vital information 110, 110′, 110″ are shown. However, it is also possible to display only one item of vital information, which can convey information about a plurality of vital signs. According to FIG. 3, for example, one numerical value is displayed, representing the pulse, heart rate and oxygen saturation. The view according to FIG. 3 may correspond, for example, to what the user can see according to FIG. 2. The field of view detection unit 112 thus detects that the patient 126 is in the field of view 108.

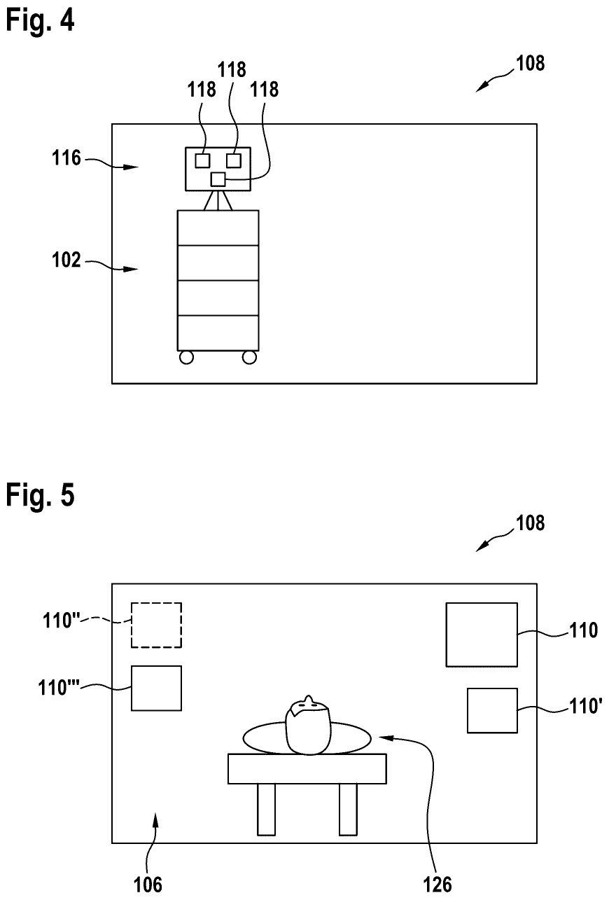

In FIG. 4, the field of view 108 is once again shown schematically from the perspective of the user 124. However, the user 124 is now looking toward the patient monitoring unit 102, which comprises the display device 116 on which vital information images 118 is displayed. This is detected by the field of view detection unit 112. The vital information images 118 represent at least substantially the same information as the vital information 110, 110′, 110″ displayed according to FIG. 3. Consequently, vital information will not be displayed.

FIG. 5 shows the same view that the user 124 has of the environment 106 as that shown in FIG. 3. The patient 126 is thus again arranged in the field of view 108. However, a critical vital condition of the patient 126 was detected by means of the patient condition detection unit 140. To alert the user 124 and to highlight important vital signs information, the vital information 110 displayed is enlarged on the basis of a patient condition parameter on which it is based. The vital information 110′ is displayed without being changed, as it is still in principle important, but is of secondary importance compared to the vital information 110′ displayed. The vital information 110″ is displayed in a manner in which it flashes on the basis of the patient condition parameter. It is also conceivable to enhance the visual effect, for example by increasing the contrast and/or the color of the displayed vital information. In addition, the patient condition parameter 110″″ is displayed so that the user 124 receives additional important information about the vital condition. The vital information displayed 110, 110′, 110″, 110″″ are visible to the user 124 in a manner in which they are graduated with respect to one another. The graduation is based on the importance of the vital signs forming the basis of the vital information 110, 110′, 110″, 110″″ displayed.

FIG. 6 is a schematic view of the medical system 100 while carrying out an intubation process. FIG. 6 is very similar to FIG. 1. Therefore, the differences shall primarily be discussed. The user 124 is wearing the portable display 104′, which comprises the field of view detection unit 112 and the patient condition detection unit 140. The user is shown performing a procedure. The procedure is an intubation procedure. The user 124 is thus currently inserting a tube 121 into the patient's trachea. To do this, they use a video stylet to which the tube 121 is attached. The video stylet is a medical imaging apparatus 120. The video stylet can be used to create images of the trachea so that the tube 121 can be placed more effectively. Image data generated by the video stylet are wirelessly transmitted to the display device 116. The display device 116 generates an image 122 for the user on the basis of the image data. The user can therefore look at the display device 116 to estimate a position of the tube 121 within the trachea, for example. By means of the procedure step detection unit 150, one of a plurality of steps for carrying out the intubation process is detected and a corresponding procedure step parameter is created. Using this parameter, the portable display 104′ displays the vital information that is important for the corresponding step. Different vital signs are important in different steps. Below is an example list of steps for performing the intubation procedure. For each step, important vital signs are listed to which at least one item of vital information displayed relates.

1. Preparation of the patient:
Important vital signs: No specific vital signs in this step. No vital information is therefore displayed.
2. Insertion of the video stylet:
Important vital signs: heart rate, blood pressure.
3. Identification of the larynx:
Important vital signs: heart rate, blood pressure.
4 Insertion of the tube 121:
Important vital signs: Heart rate, blood pressure, oxygen saturation.
5. Securing the tube 121:
Important vital signs: Oxygen saturation, carbon dioxide in the respiratory gases.
6. Completion and checking:
Important vital signs: Heart rate, blood pressure, oxygen saturation, carbon dioxide in the respiratory gases.

In particular, during completion and checking, the user 124 can therefore look at the patient 126 and has the correspondingly important vital information displayed. This is done automatically by detecting the corresponding step.

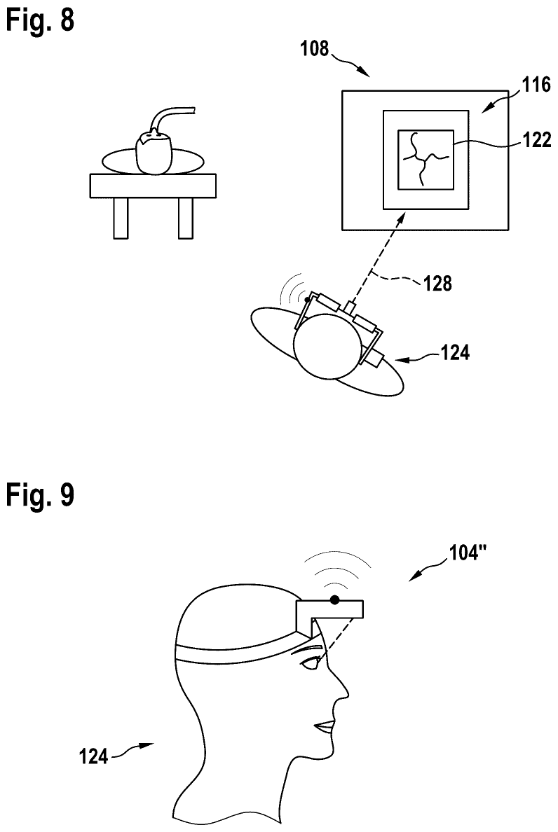

In addition to the procedure step parameter, the portable display 104′ may or may not display the vital information on the basis of the field of view parameter. Reference is made to FIGS. 7 and 8 in this respect.

If the user 124 looks in the direction of the patient 126, on whom they are performing the intubation using the imaging device 120 and the tube 121 so that said patient is in the user's field of view 108, at least one item of vital information 110 is displayed. A second piece of vital information 110′ may be displayed if this is provided according to the procedure step parameter. The display device 116 on which the image 122 is displayed is not in their field of vision. This is detected, among other things, by determining the viewing direction 128.

However, if the user 124 turns their gaze toward the display device 116, as shown in FIG. 8, vital information is not displayed. Based on the viewing direction 128, among other things, it is detected that the display device 116 and the image 122 are in the user's field of vision 108.

FIG. 9 is a schematic view of another embodiment of a portable display 104″. The portable display 104″ is a retina projector configured to project an image and/or displayed vital information onto the retina of the user 124. The portable display 104″ may be attached to a headband so that it can be worn on the head of the user 124. Alternatively or additionally, the portable display 104″ can be attached to the user's optical glasses. The portable display 104″ may have at least substantially the same functionality and/or features as the displays 104, 104′.

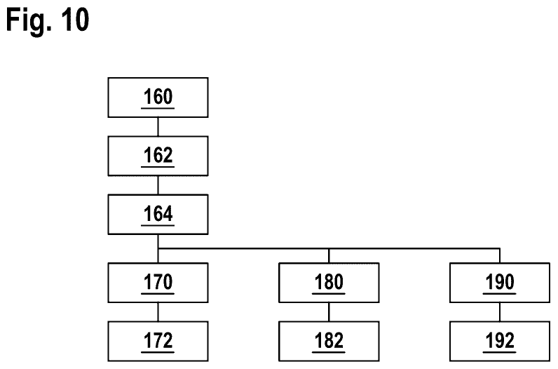

FIG. 10 shows a schematic flow diagram of a method, in particular carried out by means of a medical system 100 according to at least one of the aspects described herein and/or a portable display 104, 104′, 104″ according to any one of the aspects described herein.

The method comprises the step 160 of recording a vital sign of a patient 126, the step 162 of providing a portable display 104, 104′, 104″ for a user 124, which can be worn on a head of a user 124 and which, when worn, provides the user 124 with an at least substantially unobstructed view of an environment 106 within a field of view 108 of the user 124, and the step 164 of displaying vital information 110, which relates to the vital sign, in the field of view 108 of the user 124 by means of the portable display 104 when the display 104 is worn. Depending on which detection units 112, 140, 150 are provided, the method comprises at least one step 170 of determining a field of view parameter that relates to a current field of view 108 of the user 124, the step 172 of displaying or not displaying the vital information 110 on the basis of the field of view parameter, the step 180 of determining a patient condition parameter that relates to a current vital condition of the patient 126, the step 182 of displaying or not displaying the vital information 110 on the basis of the patient condition parameter, the step 190 of determining a procedure step parameter that describes which step of a plurality of steps of a procedure to be carried out by the user 124 on the patient 126 the user 124 is currently performing, and/or the step 192 of displaying or not displaying the vital information 110 on the basis of the procedure step parameter.

The disclosure relates, among other things, to the following aspects:

Aspect 1. A medical system (100), comprising:

a patient monitoring unit (102) configured to record a vital sign of a patient (126);

a portable display (104), which can be worn on the head of a user (124) and which, when worn, provides the user (124) with an at least substantially unobstructed view of an environment (106) within a field of vision (108) of the user (124), wherein the portable display (104) is designed to display vital information (110) within the field of vision (108) of the user (124) when worn, which relates to the vital sign; and

a field of view detection unit (112) configured to determine at least one field of view parameter relating to a current field of view (108) of the user (124);

wherein the portable display (104) is configured to either display or not display the vital information (110) on the basis of the field of view parameter.

Aspect 2. The medical system (100) according to aspect 1,

wherein the field of view parameter comprises information relating to the contents (114) of the field of view (108) of the user (124).

Aspect 3. The medical system (100) according to aspect 2,

further comprising a display device (116) configured to display a vital information image (118) that relates to the vital sign,

wherein the portable display (104) is configured not to display the vital information (110) if the field of view parameter indicates that the display device (116) is located within the field of view (108) of the user (124).

Aspect 4. The medical system (100) according to any of the preceding aspects,

further comprising:

a medical imaging instrument (120) configured to generate image data and transmit the image data;

a display device (116) configured to receive the image data and generate an image (122) for a user (124);

wherein the portable display (104) is configured not to display the vital information (110) if the field of view parameter indicates that the display device (116) is located within the field of view (108) of the user (124).

Aspect 5. The medical system (100) according to any of the preceding aspects,

wherein the portable display (104) is configured to display the vital information (110) if the field of view parameter indicates that the patient (126) is within the field of view (108) of the user (124).

Aspect 6. The medical system (100) according to any of the preceding aspects,

wherein the field of view detection unit (112) is arranged on the portable display (104).

Aspect 7. The medical system (100) according to any of the preceding aspects,

wherein the field of view detection unit (112) is arranged separately from the portable display (104).

Aspect 8. The medical system (100) according to any of the preceding aspects,

wherein the field of view detection unit (112) is configured to determine the viewing direction (128) of the user (124); and

wherein the field of view parameter relates to the determined viewing direction (128).

Aspect 9. The medical system (100) according to any of the preceding aspects,

wherein the portable display (104) can be integrated into optical corrective glasses (130).

Aspect 10. The medical system (100) according to any of the preceding aspects,

wherein the portable display (104) comprises an alarm device (132) configured to alert the user (124) if at least one of the vital signs is within a critical range.

Aspect 11. The medical system (100) according to any of the preceding aspects,

wherein the portable display (104) is configured to display the vital information (110) independently of the field of view parameter if at least one of the vital signs is within a critical range.

Aspect 12. A portable display (104) for a medical system (100) according to any of the preceding aspects.

Aspect 13. A portable display (104), in particular for a medical system (100) according to any of aspects 1 to 11, which can be worn on the head of a user (124) and which, when worn, provides the user (124) with an at least substantially unobstructed view of an environment (106) within a field of vision (108) of the user (124), wherein the portable display (104) is designed to display vital information (110), which relates to a vital sign, within the field of vision (108) of the user (124) when worn; and

comprises a field of view detection unit (112) configured to determine at least one field of view parameter relating to the current field of view (108) of the user (124);

wherein the portable display (104) is configured to display and/or not display the vital information (110) on the basis of the field of view parameter.

Aspect 14. A method for operating a medical system (100) according to any of aspects 1 to 11 and/or a portable display (104) according to aspect 12 or aspect 13.

Aspect 15. A method, in particular carried out by a medical imaging device (100) according to any of aspects 1 to 11 and/or a portable display (104) according to aspect 12 or aspect 13, comprising the steps of:

recording a vital sign of a patient (126);

providing a portable display (104) for a user (124) which is wearable on the head of a user (124) and which, when worn, provides the user (124) with an at least substantially unobstructed view of an environment (106) within a field of vision (108) of the user (124);

displaying vital information (110), which relates to the vital sign, within the field of view (108) of the user (124) by means of the portable display (104) when the display (104) is being worn;

determining a field of view parameter relating to a current field of view (108) of the user (124); and

displaying or not displaying the vital information (110) on the basis of the field of view parameter.

Aspect 16. A medical system (100), comprising:

a patient monitoring unit (102) configured to record a vital sign of a patient (126);

a portable display (104) which can be worn on the head of a user (124) and which, when worn, provides a user (124) with an at least substantially unobstructed view of an environment (106) within a field of view (108) of the user (124), wherein the portable display (104) is configured, when worn, to display a piece of vital information (110) in the field of view (108) of the user (124) which relates to the one vital sign; and

a procedure step detection unit (150) configured to determine at least one procedure step parameter describing which step of a plurality of steps of a procedure to be performed by the user (124) on the patient (126) the user (124) is currently performing;

wherein the portable display (104) is configured to display and/or not display the vital information (110) on the basis of the procedure step parameter.

Aspect 17. The medical system (100) according to aspect 16,

wherein the patient monitoring unit (102) is configured to record different vital signs;

wherein the portable display (104) is configured to display a plurality of different pieces of vital information (110), which relate to different vital signs, within the field of view (108) of the user (124) when worn;

and wherein the portable display (104) is configured to display at least one of the plurality of different items of vital information (110) on the basis of the particular step of the procedure to be performed that is detected by the procedure step detection unit (150).

Aspect 18. The medical system (100) according to aspect 16 or aspect 17,

wherein the portable display (104) is configured to visually enhance the vital information (110) displayed on the basis of the procedure step parameter.

Aspect 19. The medical system (110) according to any of the preceding aspects, in particular according to any of aspects 16 to 18,

wherein the portable display (104) is configured to enlarge the vital information (110) displayed on the basis of the procedure step parameter.

Aspect 20. The medical system (110) according to any of the preceding aspects, in particular according to any of aspects 16 to 19,

wherein the portable display (104) is configured to display the vital information (110) so as to flash, at least in part, on the basis of the procedure step parameter.

Aspect 21. The medical system (110) according to any of the preceding aspects, in particular according to any of aspects 16 to 20,

wherein the portable display (104) is configured to display the vital information (110) independently of a viewing direction (128) of the user (124).

Aspect 22. The medical system (100) according to any of the preceding aspects, in particular according to any of aspects 16 to 21,

wherein the procedure step detection unit (150) comprises a camera (134) configured to generate image data, and

wherein the procedure step detection unit (150) is configured to determine the at least one procedure step parameter using the image data.

Aspect 23. The medical system (100) according to aspect 22,

wherein the camera (134) is arranged on the portable display (104).

Aspect 24. The medical system (100) according to aspect 22,

wherein the camera (134) is arranged separately from the portable display (104).

Aspect 25. The medical system (100) according to any of the preceding aspects, in particular according to any of aspects 16 to 24,

wherein the procedure is the intubation of the patient (126).

Aspect 26. The medical system (100) according to any of the preceding aspects, in particular according to any of aspects 16 to 25, further comprising:

a patient condition detection unit (140) configured to determine at least one patient condition parameter relating to a current vital condition of the patient (126).

Aspect 27. The medical system (100) according to aspect 26,

wherein the portable display (104) is configured to either display or not display the vital information (110) on the basis of the procedure step parameter and the patient condition parameter.

Aspect 28. The medical system (100) according to aspect 27,

the portable display (104) is configured to, when the patient condition parameter indicates a critical vital condition of the patient (126), either display or not display the vital information (110) independently of the procedure step parameter.

Aspect 29. The medical system (110) according to any of the preceding aspects, in particular according to any of aspects 16 to 28,

wherein the portable display (104) can be integrated into optical corrective glasses (130).

Aspect 30. A portable display (104) for a medical system (100) according to any of the preceding aspects, in particular according to any of aspects 16 to 29.

Aspect 31. A portable display (104), in particular for a medical system (100) according to any of aspects 16 to 29, which can be worn on the head of a user (124) and which, when worn, provides a user (124) with an at least substantially unobstructed view of an environment (106) within a field of vision (108) of the user (124), wherein the portable display (104) is designed to display vital information (110), which relates to the one vital sign, within the field of vision (108) of the user (124) when worn; and

comprises a procedure step detection unit (150) configured to determine at least one procedure step parameter describing which step of a plurality of steps of a procedure to be performed by the user (124) on the patient (126) the user (124) is currently performing;

wherein the portable display (104) is configured to display and/or not display the vital information (110) on the basis of the procedure step parameter.

Aspect 32. A method for operating a medical system (100) according to any of aspects 16 to 29 and/or a portable display (104) according to aspect 30 or aspect 31.

Aspect 33. A method, in particular carried out by a medical imaging device (100) according to any of aspects 16 to 29 and/or a portable display (104) according to aspect 30 or aspect 31, comprising the steps of:

recording a vital sign of a patient (126);

providing a portable display (104) for a user (124) which is wearable on the head of a user (124) and which, when worn, provides the user (124) with an at least substantially unobstructed view of an environment (106) within a field of vision (108) of the user (124);

displaying vital information (110), which relates to the vital sign, within the field of view (108) of the user (124) by means of the portable display (104) when the display (104) is being worn;

determining a procedure step parameter that describes which step of a plurality of steps of a procedure to be performed by the user (124) on the patient (126) the user (124) is currently performing; and

displaying or not displaying the vital information (110) on the basis of the procedure step parameter.

1. A medical system, comprising:
a patient monitoring unit configured to record a vital sign of a patient;
a portable display which can be worn on the head of a user and which, when worn, provides a user with an at least substantially unobstructed view of an environment within a field of view of the user, wherein the portable display is configured, when worn, to display a piece of vital information in the field of view of the user which relates to the one vital sign; and
a patient condition detection unit configured to determine at least one patient condition parameter relating to a current vital condition of the patient;
wherein the portable display is configured to either display or not display the vital information on the basis of the patient condition parameter.

2. The medical system as set forth in claim 1, wherein the portable display is configured to display the vital information if the at least one patient condition parameter is within a critical range, and
wherein the portable display is configured not to display the vital information if the at least one patient condition parameter is not within the critical range.

3. The medical system as set forth in claim 1, wherein the portable display comprises an alarm device configured to alert the user if the at least one patient condition parameter is within a critical range.

4. The medical system as set forth in claim 3, wherein the portable display is configured to alert the user by means of a vibration alarm.

5. The medical system as set forth in claim 3, wherein the portable display is configured to alert the user by means of an acoustic alarm signal.

6. The medical system as set forth in claim 1, wherein the portable display is configured to visually enhance the vital information displayed on the basis of the patient condition parameter.

7. The medical system as set forth in claim 1, wherein the portable display is configured to enlarge the vital information displayed on the basis of the patient condition parameter.

8. The medical system as set forth in claim 1, wherein the portable display is configured to display the vital information displayed in a manner in which it flashes, at least in part, on the basis of the patient condition parameter.

9. The medical system as set forth in claim 1, wherein the portable display is configured to display the vital information independently of a viewing direction of the user.

10. The medical system as set forth in claim 1, wherein the portable display can be integrated into optical corrective glasses.

11. A portable display configured to communicate with a patient monitoring unit, the patient monitoring unit configured to record a vital sign of a patient, the portable device configured to worn on the head of a user and, when worn, provides a user with an at least substantially unobstructed view of an environment within a field of vision of the user, wherein the portable display is configured to display a vital information which relates to the vital sign, within the field of vision of the user when worn, the portable display comprising:
a patient condition detection unit configured to determine at least one patient condition parameter relating to a current vital condition of the patient;
wherein the portable display is configured to either display or not display the vital information on the basis of the patient condition parameter.

12. A method carried out by means of the medical system set forth in claim 1, comprising the steps of:
recording a vital sign of a patient;
displaying vital information, which relates to the vital sign, within the field of view of the user by means of the portable display when the display is being worn;
determining a patient condition parameter relating to a current vital condition of the patient; and
displaying or not displaying the vital information on the basis of the patient condition parameter.
